(19) United States
(12) Patent Application Publication
(10) Pub. No.: US 2011/0245104 A1
(43) Pub. Date: Oct. 6, 2011

(54) Method for screening the sensitizing properties of chemical compounds

(76) Inventors: Kerstin S. Broo, Lindome (SE); Brigitte Bauer, Goteborg (SE); Marica B. Ericson, Goteborg (SE); Anna-Lena Stenfeldt, Goteborg (SE); Sofia I. Andersson, Goteborg (SE)

(21) Appl. No.: 13/065,692

(22) Filed: Mar. 28, 2011

Related U.S. Application Data
(60) Provisional application No. 61/341,355, filed on Mar. 29, 2010.

(51) Int. Cl. C40B 30/06; C12Q 1/02; G01N 33/53; G01N 33/573; C12Q 1/68

(52) U.S. Cl. 506/10; 435/29; 435/7.1; 435/7.4; 204/456; 435/6.1

(57) ABSTRACT

A method is provided for screening the sensitizing properties of chemical compounds. The method is based on keratinocytes or cells that share important hallmarks of these cells, but other components e.g. proteins could also be used. This method is of importance for several conditions, including but not limited to allergic contact dermatitis (ACD), drug hypersensitivity reactions (DHRs) and autoimmune diseases.

### CROSS-REFERENCE TO RELATED APPLICATIONS

This application claims priority from U.S. Provisional Application Ser. No. 61/341,355 filed Mar. 29, 2010.

### FIELD OF THE INVENTION

The present invention relates generally to immunology. More specifically the invention relates to a method for screening the sensitizing properties of chemical compounds. This method is of importance for several conditions, including but not limited to allergic contact dermatitis (ACD), drug hypersensitivity reactions (DHRs) and autoimmune diseases.

### BACKGROUND OF THE INVENTION

The skin is the principal barrier between self- and non-self and it protects us from dehydration and from harmful effects of exposure to for example microorganisms, chemicals, and UV radiation. Sometimes the skin fails to protect against the compounds around us and that can result in, among other things, irritant contact dermatitis (ICD) or allergic contact dermatitis (ACD). ACD is a permanent, specific immunologic hypersensitivity reaction, whereas ICD is a non-immunological local inflammatory reaction. ACD affects 15-20% of the population in the Western world and it is the most prevalent form of immunotoxicity found in humans. As ACD is a common occupational disease, there are substantial psychosocial and socioeconomic effects.

ACD is caused by small molecules (haptens) that enter the skin barrier. Once an individual is sensitized, there is today no cure, but the offending compound and any cross-reacting compounds will have to be avoided. This can be very difficult to do for commonly occurring allergens such as nickel, preservatives and fragrances. The growing list of compounds that can cause ACD currently comprises >4000 substances. In order to predict the sensitizing properties of a compound, the OECD approved benchmark test today is the murine Local Lymph Node Assay (LLNA) (The local lymph node assay and the assessment of relative potency: status of validation. Basketter D A, Gerberick F, Kimber I. Contact Dermatitis. 2007 August; 57(2):70-5). However, according to EU directives, a ban on in vivo testing of cosmetic and toiletry ingredients will be in place from 2013. Hence, there is a need for an alternative that can replace this benchmark test.

The skin is a stratified tissue, and the outermost layer is called the epidermis. The epidermis, in turn, can be subdivided into a dead layer (the stratum corneum, SC) and the living epidermis. The epidermis is mainly (˜95%) composed of keratinocytes. These are interspersed with the specialized antigen-presenting cells (Langerhans cells), Merkel cells and melanocytes. Keratinocytes get their name from their large amount of type I and II intermediate filaments called keratins. These structural proteins are essentially what give the epidermis (and epithelia) high tensile strength. Epithelial tissues express different pairs of keratins depending on the cell type and the stage of differentiation. The keratinocytes in the basal layer of stratified epithelia express chiefly the keratin 5/keratin 14 (K5/K14) pair and up to 35% of the protein content is K5 and K14. This pair is replaced by the K1/K10 pair in the suprabasal layers, which accounts for as much as ˜80-85% of the total cellular content of a fully differentiated keratinocyte. In summary, the epidermis is mainly composed of keratinocytes that in turn have a very high content of keratins.

Keratins form bundles of insoluble filaments to afford an intracellular skeleton. When cells in the basal layer (the only mitotic layer in the epidermis) go through apoptosis (as a part of normal growth regulation), there is a mechanism in place to deal with the insoluble keratins that would otherwise form amyloid plaques. Basal keratinocytes that go through apoptosis thus dispel keratins, along with other cellular remnants, in apoptotic blebs. In skin, these are called keratin bodies. Keratin bodies drop through the basement membrane and are then phagocytosed by dendritic cells.

ACD has been shown to be mediated by T-cells. T-cell mediation comes about through MHC (major histocompatibility complex) restriction and presentation of peptides to naïve T-cells in the lymph nodes. Since MHC molecules present peptides on the order of 8-10 amino acids (MHC class I), or 9-25 amino acids (MHC class II), a hapten in itself is thus too small to warrant a T-cell mediated immune response. Instead, in a seminal paper published as early as 1935 by Landsteiner and Jacobs, the key step in sensitization is proposed to be the formation of the complete allergen through covalent linkage of the hapten to a carrier protein turning self into non-self. Inspection of the list of known haptens reveals common traits: haptens are typically small (≦1000 Da), hydrophobic (log P≧2), and they frequently contain an electrophilic group. The covalent linkage that turns self into non-self is therefore primarily envisioned as a reaction between an electrophilic group on the hapten and a nucleophilic moiety in a protein. Alternatively, in the case of haptens containing not one (monodentate) but two reactive groups (bidentate), a cross-linking of two self-proteins could also occur. Seemingly innocent compounds that do not contain electrophilic moieties can also be allergenic. It has been proposed that these compounds can be autoxidized or photo-oxidized outside the body (prehaptens) or metabolized in the skin (prohaptens) to form potent haptens.

The currently accepted theory is that a compound causes ACD through the following steps:
Penetration of the compound into the epidermis where it conjugates to proteins. Uptake/processing of haptenated proteins by skin-residing dendritic cells. The focus has been on the Langerhans cells of the epidermis, but dermal dendritic cells have been gaining more attention lately. Subsequent hapten-induced maturation and migration of Langerhans cells. Mature Langerhans cells present the MHC class I or II restricted (depending on the nature of the hapten) haptenated peptide to naïve T cells in the draining lymph nodes. Helper T cells (T_{H}) and/or cytotoxic T cells (T_{C}) proliferate and enter the circulation. The affected person is now sensitized.
The next time the hapten is encountered; the allergy is elicited.

The only way to avoid the allergic symptoms is henceforth to avoid the hapten and any cross-reacting compounds.

As stated above, the key step in sensitization is proposed to be the formation of complete allergen through the covalent linkage of the hapten to a carrier protein thus turning self into a non-self. To decode sensitization, haptenated proteins need to be singled out with molecular precision against the entire backdrop of the proteome ending up with identification of the targeted protein(s) and the exact position of haptenation. This has not been previously achieved.

Drug hypersensitivity reactions (DHRs) are a major problem and the mechanisms behind DHR are not known to a full extent. DHRs represent approximately ⅓ of all adverse drug reactions; are sometimes life threatening; can require hospitalization; and entail changes in drug prescription. This is an important public health problem, since more than 7% of the population is concerned. DHRs are unfortunately unpredictable and prospective clinical studies are very difficult to perform. The use of animal models (rats, guinea pigs, cats, dogs) is almost the only way to test the likelihood of a compound to cause DHRs. However, because DHRs are also unpredictable in animals, it is difficult to obtain and know beforehand if a certain animal model will correctly predict the effect in humans.

The invention may be used in risk assessment for allergic contact dermatitis. In order to predict the sensitizing properties of a compound (i.e. the capacity to induce the adaptive immune response through skin contact), the benchmark test today is the OECD approved LLNA. According to the EU directives, a ban on in vivo testing of cosmetic and toiletry ingredients will be in place from 2013. Unfortunately, without a molecular understanding of how the immune system is triggered by haptens, there can be no in vitro or in silico (computer) models that can safely replace the LLNA. In order to replace an animal, a large number of steps occurring in a complex biological setting will have to be mimicked in vitro. In an EU initiative, SENS-it-ive, primarily by cell biologists and immunologists, a large number of research groups are working towards an in vitro replacement of the LLNA. Their work packages detail a continuum/extension of strategies similar to previously published reports, i.e. global genome analyses, immunofluorescence assays, etc. The European trade association for the cosmetic, toiletry and perfumery industry (COLIPA) has a similar task force and strategy. The common drawback in all of these attempts is that the wrong type of cell that does not express the major hapten targets has been used. When keratinocytes have been present in a cellular test in the prior art, they have mostly been viewed as providing inflammatory signals.

In lieu of a detailed understanding of the governing processes behind ACD, all major efforts towards creating in vitro replacements have so far focused on the antigen-presenting cells of the epidermis (Dendritic cells and skin sensitization: biological roles and uses in hazard identification. Ryan C A, Kimber I, Basketter D A, Pallardy M, Gildea L A, Gerberick G F. Toxicol Appl Pharmacol. 2007 Jun. 15; 221(3):384-94). This has been in accordance with the accepted theories (vide supra). The present invention will show that this is the wrong cell-type on which to focus.

Another major drawback is that readouts have not been homogeneous and not conducted in living cells in real time. Typical tests use RT-PCR in cell lysates and/or immunofluorescence assays. However, an ELISA test might take 2 days to complete, precluding investigations/optimizations of hapten concentrations and time points. The time demanding, numerous washing and (antibody) treatment steps may also introduce experimental artifacts and errors. A whole-cell assay enables quick measurements without introducing experimental artifacts. In addition, in a high-throughput screen, a considerable amount of specific antibodies must be used which is resource- and cost-demanding, and it does involve the use of animals to obtain antibodies. The screening method of the present invention is based on the blebbing of living cells, providing read-out in real-time, without the use of antibodies, and is directly amenable to scale-up in a high throughput fashion.

In other previous attempts at cell-based tests it has been standard procedure to use a concentration of hapten that results in >80% cell (dendritic cells/cell lines) viability instead of using standardized concentrations of haptens. This is done to avoid cytotoxic effects. This has the result of introducing two major drawbacks besides the use of the wrong cell type. First, the sensitization is related to a large number of keratinocytes dying causing the expulsion of membrane blebs. Thus a cell assay based on keeping a high viability will lead to erroneous results. The test method of the present invention is instead based on the processes that occur in the body, where the immune system is exposed to large amounts of cryptic epitopes and neoepitopes (differently processed and/or haptenated). Second, it makes it very hard/impossible to compare substances with each other. It is also virtually impossible to normalize the response of substance A with a control substance or compare A with substance B if all are at different concentrations and/or at different time points (as in many previous cell-based tests). The present test method uses standardized concentrations of test substances, enabling direct comparisons and standardizations. These are vital features in obtaining a graded response (vide infra).

In summary, until now it has not been possible to accurately mimic the sensitization process in an in vitro, cell-based process. Current attempts for in vitro tests are not based on the processes occurring in the body. However, herein is presented a predictive, cell-based, in vitro test of the sensitizing potential of compounds based on findings of the actual mechanisms taking place in the body during sensitization. Human keratinocytes are used as the basis of the test and the blebbing response is measured when compounds are added to living keratinocytes. The blebbing response (with grading and measured outputs, etc.) can also be used to measure the risk of DHRs, autoimmune diseases, and other conditions caused by epithelial contact with different compounds, mixtures, formulations, and solutions.

### SUMMARY OF THE INVENTION

An object of the present invention is to use the knowledge of keratinocytes' role in allergic contact dermatitis (ACD) to create new ways of screening compounds for ACD.

Another object is to screen compounds for ACD using keratinocytes and analysis of keratins and other proteins.

Another object is to use the concept of blebs in an in vitro test screening for ACD.

Another object is to provide products and kits for screening different compounds for ACD.

Another object is to provide products and kits for screening different compounds that inhibit the blebbing response, to prevent sensitization and/or development of autoimmune diseases.

Another object is to provide products and kits for screening different compounds that inhibit the blebbing response, to prevent the release of blebs after exposure to drug formulations.

Another object is to use the invention to screen compounds for risk assessment in allergies contracted through contact with epithelial cells (oral, eyes, through inhalation, etc.).

Another object is to use the invention to screen compounds for risk assessment in drug-induced hypersensitivity and autoimmune diseases.

Another object is to use the invention to screen compounds for the possibility of inhibiting blebbing responses.

Another object of the invention is to use the molecules found using the invention that inhibit the blebbing (i.e. the inhibitors).

Another object of the invention is to use structural analogs of the identified inhibitors.

Other objects and advantages of the present invention will become obvious to the reader and it is intended that these objects and advantages are within the scope of the present invention.

### BRIEF DESCRIPTION OF THE DRAWINGS

FIG. 1. A) Is a schematic drawing that illustrates the findings herein on what happens when sensitizers come into contact with the skin. B) Is a schematic drawing that illustrates the blebbing response exhibited by keratinocytes when they are exposed to sensitizers/test compounds in vitro.

### DETAILED DESCRIPTION OF THE INVENTION AND PREFERRED EMBODIMENTS THEREOF

### Definitions

As used herein the terms “keratinocytes” or “keratinocyte cells” are intended to mean primary human keratinocytes, or keratinocytes from other mammals. The term may further include primary, immortalized and stem cell culture systems such as adult primary human epidermal keratinocytes (HEKa) or neonatal primary human epidermal keratinocytes (HEKn)s or cell lines with HEKn characteristics, such as HaCaT.

As used herein the terms “keratin” or “keratins” are intended to mean the keratin protein family of fibrous structural proteins being one of the key structural components of skin, hair and nails. The term is predominantly intended, but not limited, to mean the specific keratin subtypes keratin 1, 5, 8, 10, 14 and 18.

As used herein the term “hapten” is intended to mean a xenobiotic compound that can form immunogenic complexes with endogenous macromolecules, e.g. proteins, in the human body and activate the adaptive immune system. Generally the term refers to a xenobiotic compound able to cause e.g. contact allergy when breaching the skin barrier, reacting with proteins therein, but may also refer to compounds with other entrance routes e.g., but not limited to subcutaneously, intravenously, per orally.

As used herein the term “haptenation” is intended to mean the reaction and complex formation between a hapten and a macromolecule, e.g. a protein, and “haptenated protein” is intended to mean the formed hapten-protein complex, i.e. a protein that has bound a hapten.

As used herein the term “haptenation site” or “haptenation location” is intended to mean a specific amino acid of the specific protein with which the hapten reacts, i.e. the exact location of the hapten on the protein.

As used herein the terms “blebs” or “blebbing” are intended to mean the formation and release of membrane vesicles from cells. Blebbing is the process where membrane protrusions are formed. Blebs are protrusions formed at plasma membrane of a cell, which eventually dissociate or bud off from the cell into the surrounding medium. Blebs are in general of spherical nature and consist of the plasma membrane bilayer with a diameter of about 1 μm to about 100 μm. The term “keratinocyte blebbing response” is intended to mean the cellular expulsion of membrane lipid vesicles specifically from keratinocytes or keratinocyte-type cells described previously.

As used herein the term “sensitization” is intended to mean the immunological process by which an individual is exposed to e.g. a contact allergenic compound, i.e. a hapten that by forming immunogenic complexes with macromolecules activates the adaptive immune system.

As used herein the term “elicitation” is intended to mean the cutaneous inflammatory reaction of an individual due to re-exposure of a compound to which the individual is sensitized or due to the exposure of any crossreacting compounds.

As used herein the term “protein derivatization” is intended to mean the event of a compound (e.g. a hapten) binding to a protein.

As used herein the term “allergic contact dermatitis (ACD)” is intended to mean the clinical manifestation of the cutaneous immunological hypersensitive reaction that an individual develops when re-exposed to a contact allergen (hapten) to which the individual is sensitized, or any crossreacting compounds.

As used herein the terms “drug hypersensitivity reaction (DHRs)” or “protein drug-induced hypersensitivity reactions” are intended to mean an immune-mediated reaction to a drug, in which the drug act as hapten, binding covalently endogenous proteins causing sensitization, and at subsequent re-exposure, elicitation.

As used herein the term “autoimmune diseases” is intended to mean a disease in which the immune response reacts against endogenous substances, constituents and tissues normally present in the body. This will include, but is not limited to diseases such as rheumatoid arthritis, SLE (systemic lupus erythematosus), Sjögrens Syndrome (SS), type I diabetes and Psoriasis.

As used herein the term “standard sensitizer” is intended to mean a compound known to cause contact allergy. This will include, but is not limited to compounds normally present in the standard patch test series for allergy testing, e.g. formaldehyde, fragrance mix, and parabens.

As used herein the term “confluency” is intended to mean the complete or almost complete (at least approximately 50-60% coverage) coverage of the cell culture dish or the flask or another material by the cells.

As used herein the terms “cryptic epitopes and “neoepitopes” are intended to mean haptenated peptides and proteins, and/or proteins and peptides that have not been processed as they normally would have been, and or cross-linked proteins. These epitopes are thus normally concealed from the immune system.

As used herein the term “epitope spreading” is intended to mean the development of immune responses to epitopes distinct from, and noncross-reactive with, the dominant epitope.

Contrary to common belief, the present inventors have surprisingly found that in human ACD, keratinocytes play a crucial role in the sensitization process. The evolutionary defense systems of human skin are aimed at large parasites, viruses and microorganisms. However, it can be shown that this results in ideal, stratified physio-chemical properties that allow penetration of reactive haptens down to, and into the basal keratinocytes. Furthermore, it has been found that, in addition to other proteins, important targets are keratins, and specifically keratins 5 and 14 (K5 and K14) in human skin excerpts. In particular, the amino acid cysteine 54 (C54) of K5 has been identified as a target for thiol-reactive haptens in human tissue and primary human keratinocytes. This residue is situated in a stretch of amino acids that makes crucial non-covalent contacts with the C-terminal tail of desmoplakin, thereby anchoring the cell with its surroundings through desmosomes (cell-cell) and hemidesmosomes (cell-basement membrane). An alteration of nearby residues has been shown to substantially reduce the keratin-desmoplakin interaction, thereby reducing the cell-cell and cell-matrix interactions, resulting in a homeless cell. If an epithelial cell loses its contacts with its surrounding, a growth regulation process is in place to prevent erroneous colonization. Thus, a homeless epithelial cell is doomed to anoikis, i.e. apoptosis brought on by “being without a home”. During apoptosis, membrane vesicles, so-called “blebs”, may appear on the surface of the cells. The cell then breaks down into smaller fragments called apoptotic bodies. Phagocytic cells then engulf the apoptotic bodies without causing an inflammatory reaction. Blebbing is a common phenomenon; in addition to apoptosis it happens for example during the cytokinesis phase of cell division, in nonmotile embryonic blastomeres, and in some cell migration events.

Through studies on human keratinocytes, it can be shown that haptens covalently modify keratins (and other proteins), and that thiol-reactive haptens, such as mBBr (monobromobimane), modify C54 of K5. Furthermore, it has been shown, through studies on human keratinocytes that haptens, directed against different nucleophiles in proteins, bring on blebbing, thus releasing cryptic epitopes and neoepitopes (e.g. haptenated peptides and proteins, and/or proteins and peptides that have not been processed as they normally would have been). Through Western blot experiments, it is shown that blebs from the predominantly amine-directed hapten oxazolone and the predominantly arginine-reactive hapten glyoxal also contain K5 and K14. Furthermore it is also shown that irritants (e.g. sodium dodecyl sulphate, SDS) and non-allergenic compounds (e.g. nonanoic acid) do not initiate blebbing. It is shown that the theory is relevant in an in vivo setting by performing ELISA experiments on sera from experimental animals (mice). In sera from animals that had been exposed to sensitizing compounds (via skin contact) antibodies against K14 were detected. Furthermore, it was found that contact with some sensitizing compounds (including but not limited to dBBr (dibromobimane)) results in increased levels of acute-phase proteins such as serum amyloid P component (SAP).). Normally, the plasma concentration of SAP increases in response to inflammation. It is known that chronic inflammation can lead to self-attack (autotoxicity) due to the innate immune defence. An increased level of SAP is a common feature in autotoxic diseases such as Alzheimer's.

Based on the literature, covalent modification, in all likelihood, short circuits defense mechanisms that prevent erroneous colonization by homeless epithelial cells. In summary, haptens penetrate down to the basal layer, enter the basal keratinocytes, react with K5 and K14 (and other, less abundant proteins), thus bringing on blebbing, resulting in the formation of keratin bodies (i.e. blebs containing among other cellular remnants, haptenated i.e. non-self proteins including keratins as well as other proteins), that drop below the basal membrane, where the neo and/or cryptic epitopes are phagocytosed by antigen-presenting cells (dendritic cells). This process thus results in exposing the immune system to a large amount of cryptic epitopes, and furthermore, some of the proteins are haptenated i.e. non-self. Haptenation could also affect the processing of proteins such that neoepitopes are exposed. All of these aspects likely contribute to sensitizing the individual. It is not excluded that smaller blebs, including, but not limited to, exosomes also contribute to sensitization. Elicitation occurs when the haptens again derivatize keratins, keratin-derived peptides and/or other proteins, probably both in the stratum corneum and in the basal layer. Other proteins may of course also be derivatized; however keratinocytes make up ˜95% of the cells in the epidermis, and keratins make up ≧80% of the total cellular protein in a fully differentiated keratinocyte. Of note is that K5 is homologous to other type II keratins such as K1, and K14 is homologous to other type I keratins such as K10. Thus, in the elicitation phase, when a compound reacts with K1 and K10 in the suprabasal layers, homologous epitopes are produced. Both in sensitization and elicitation, the body will thus be subjected to large amounts of highly homologous/identical recurring epitopes, as keratins are derivatized.

Furthermore, emerging from the present work is a theory of recurring epitopes upon epithelial exposure of chemical compounds. All stratified, squamous epithelial basal cells express the target proteins in high abundance (up to 35% of the cellular content). Epithelial cells line the cavities and surfaces of structures throughout the body and many glands are also formed from epithelial tissue. For example, K5 antibodies stain cells in tissue such as the: bronchus (respiratory epithelial cells), cervix, epididymis, esophagus, nasopharynx, oral mucosa, prostate, salivary glands, urinary bladder, squamous epithelial cells in the tonsils, vagina and vulva/anal skin. Macrophages in lung are also stained. Human corneal epithelia also stain for K5. As K14 (and K15) is expressed in a pair with K5, K14 is also present in these tissues and cells. In combination with the fact that simple epithelial cells express the homologous K8/K18 pair, the results thus indicate a general mechanism whereby the identical or highly homologous epitopes are encountered irrespective of the route of entry of the compound into the body. The use of other epithelial cells and epithelial tissue for analysis of the blebbing response is therefore within the scope of the invention.

The blebbing response can therefore be utilized as a predictive in vitro test for various ailments, including for example ACD, DHR, but also chemically induced asthma, and not excluding other ailments that can be caused through epithelial contact with a compound.

The finding that the identical or highly homologous epitopes are encountered irrespective of the route of entry of the compound into the body (exemplified by K5/K14 and K8/K18) has other implications. Thus, when epithelial contact (such as via the skin) with a compound has caused basal keratinocytes to die and release blebs, autoantibodies (and likely memory T-cells) against keratins (and other proteins) are produced, and the individual has now been sensitized. If another, unrelated compound such as a drug is ingested/inhaled/or otherwise then comes into contact with epithelial tissue, it can react with basal epithelial cells, and it too can cause blebbing. The same epitopes that the individual already has autoantibodies against are again released in the blebs. The circulating memory T-cells and/or the autoantibodies against the protein epitopes are then recruited and a drug hypersensitivity reaction (DHR) is observed. Thus, for some DHRs (including, but not limited to, fixed-drug eruptions) it is highly likely that the same mechanisms, with regards to how a compound interacts with epithelial cells, are operating. These findings explain why DHRs can be observed even when the individual never has been exposed to the drug in question before, as the response can be largely directed against the released self-proteins. This hypothesis is also corroborated by the finding that basal keratinocytes are targeted in drug-induced hypersensitivity reactions (Incidence of circulating antibodies reactive with basal cells of skin in drug reactions. T van Joost. Acta Dermatovener (Stockholm) 54: 183-188, 1974).

Thus, the blebbing response can also gauge the risk of compounds (neat, in mixtures, formulations, solutions, etc.) to cause drug-induced hypersensitivity reactions (DHRs) using relevant human epithelial cells. The invention presented herein thus represents a major advancement in predictive screening for adverse drug reactions upon epithelial contact.

Furthermore, the present inventors have shown that haptenation results in the release of cryptic epitopes, otherwise non-accessible to the immune system and/or neoepitopes that have been differently processed. This is important not only for contact allergy, but it has dire implications concerning the breach of self-tolerance in autoimmune diseases. This hypothesis is borne out by our findings of autoantibodies against K14 in murine sera. That is, the ELISA measures not antibodies against haptenated proteins, but antibodies towards the proteins themselves. Even though the experiments, for ethical reasons, are performed on mice, it is highly likely that humans will also develop autoantibodies upon skin contact with sensitizers. Many processes can occur in the body once blebbing is instigated and self-tolerance is breached (i.e. breach of tolerance against K5 and K14, breach of tolerance against other proteins as stated above, epitope spreading through homology, release of other apoptotic debris like ribonuclear proteins, etc.).

Examples of these autoimmune diseases include SLE (systemic lupus erythematosus); rheumatism (RA); Sjögrens Syndrome (SS); and type I diabetes, but do not exclude other autoimmune diseases. Notably, in these diseases (and other autoimmune diseases) autoantibodies against keratins have been reported in the literature. Also, keratinocytes are targets of autoantibodies found in sera from patients with atopic dermatitis. Thus the present findings on the blebbing response can also be used to measure the ability of compounds, mixtures, solutions and other formulations to cause breach of self-tolerance and thus prevent autoimmune diseases. Since this is a new finding and a new theory, there are currently no tests that provide this detailed understanding.

Glycine-rich cell wall protein (GRP) is a ubiquitous food protein that has a high homology with keratins and other self proteins (e.g. Epstein-Barr virus nuclear antigen-1 (EBNA-1), heterogeneous nuclear ribonucleoprotein and fibrillar collagen), which are common targets in autoimmune disorders. Antibodies against a peptide epitope derived from GRP and peptide-specific T cell clones can be found in sera from patients with autoimmune disorders (RA, SLE, Psoriatic arthritis PsA, Chronic idiopatic urticaria CIU), CPI (Chronic parvovirus B19 infection) and food allergies (cereals, fish, fruit and vegetables). However, normal subjects do not show these immune responses (“Glycine-rich cell wall proteins act as specific antigen targets in autoimmune and food allergic disorders.” C. Lunardi, et. al., International Immunology, Vol. 12, No. 5, pp 647-657).

Furthermore, the anti-GRP peptide antibodies purified from the sera specifically recognized EBNA-1 and autoantigens such as keratin, collagen and actin. Thus, antigen spreading through a common (homologous) sequence among apparently divergent proteins from plants, viruses and humans may be responsible for autoimmune diseases as well as food allergies. Importantly, it is quite conceivable that this constitutes an example of epitope spreading, not from GRP, but from keratins and other proteins released in blebs through the action of sensitizing molecules (haptens) upon epithelial exposure. The invention can therefore also be used to study the blebbing response in order to address questions related to e.g. food allergies.

The molecular understanding of what happens when compounds encounter epithelia and epithelial cells resulting in e.g. a blebbing response as presented above has made it possible to develop products for in vitro screening of e.g. compounds, mixtures, formulations, and solutions for:
their sensitizing potential in allergic contact dermatitis
other ailments caused by epithelial contact
risk of causing breach of self-tolerance, i.e. risking autoimmune diseases
risk of causing DHRs
risk of causing food allergies
risk of autotoxicity

In addition, the invention makes it possible to use the blebbing response as a means for screening the inhibition of the blebbing response by addition of other substances (henceforward called inhibitors) to e.g. mixtures, formulations, and solutions. These inhibitors (or compounds that mimic the key interactions of the found inhibitors) can then potentially be used as additives in the formulations, solutions, mixtures, etc. to prevent sensitization and the negative downstream effects of sensitization that are mentioned elsewhere in this application. This is only possible in a cell-based test that, like in the present invention, mimics the in vivo events. All of these applications of the invention are made possible through the new molecular understanding and thus represent applications that previous technologies are unable to provide.

To study the reactions occurring upon contact with epithelial tissue such as the skin the inventors have deployed caged haptens (bromobimanes) that are non-fluorescent prior to displacement of the bromine atom. It should be noted that hazardous compounds that can cause immunological problems are called “haptens” in diseases such as allergic contact dermatitis. The usage of the word “hapten” should however not be taken to exclude all other conditions that can result through epithelial contact with compounds. On the contrary, the invention also includes the usage of the blebbing response as a means to gauge the risk for a new compound, mixture, solution and other formulations to cause autoimmune diseases; DHRs; etc.

Uncaging of bromobimanes occurs preferably with thiols (cysteines) that are potent nucleophiles at physiological pH. Preferential reactivity towards thiols is advantageous since many, if not most; clinical haptens are thiol-reactive. The other major hapten-reactive moiety in peptides and proteins is the nucleophilic amine group (the —NH₂ group of lysines and the α-NH₂ group that constitutes the amino terminus of proteins). A series of bimanes with none (synBim), one (mBBr), or two reactive groups (dBBr) was deployed to make major steps forward in elucidating the detailed molecular mechanisms behind sensitization and epithelial immunotoxicity.

The first step is that the molecules come into contact with the skin (the stratum corneum). Many of the reactive molecules react in the stratum corneum, something that can be seen in experiments with human skin excerpts and the caged (mBBr and dBBr) haptens. However, a significant proportion of the molecules penetrate further down to the suprabasal layers of the epidermis but also the follicular pathway is of importance for bringing xenobiotics into the skin. It is likely that the penetration is due both to the acidic pH of the stratum corneum (the molecules react more slowly when the pH is low), but also because many of the available groups have reacted. In the suprabasal layers, the haptens have a hard time penetrating the cells, as the cornification of the cells creates a thick, relatively impermeable envelope around the suprabasal cells. Consequently, the haptens penetrate down to the basal layer. Here, the cell membrane is much thinner and more fluidic and the molecules can thus enter the basal cells. The experiments with human skin excerpts show, that in addition to the stratum corneum; cells in the basal layer are labeled by the caged haptens dBBr and mBBr.

The second step is that the molecules enter the basal cells. Since the extracellular environment is oxidizing, extracellular cysteines are mostly oxidized to disulfides. However, it is in the reduced, intracellular environment that the haptens find reduced thiols to react with. Once inside the cells the compounds will react with available (i.e. not sterically hindered), reduced cysteines (thiol groups). In this step, it is demonstrated that the labeled cells are basal keratinocytes. Furthermore, it was identified that one of the cysteines that the haptens react with is cysteine 54 (C54) of K5. Using a LTQ-FT-ICR mass spectrometer equipped with a nanospray source it was found that the thiol-reactive hapten mBBr modifies C54 of K5 in intact, full-thickness human skin and when living human keratinocytes have been exposed to mBBr. One of the reasons for intense reaction at C54 is presumably its high effective concentration just inside the cell membrane. It is vital in the life of an organism to fail-safe its epithelial integrity, and the bundling of the KIFs ensures a high effective local concentration of the peptide stretch containing C54, ensuring strong welding of the cell to its surroundings. Amino acids nearby in the primary sequence are consequently also located within physical reach of an incoming compound in similarly high effective concentrations. Looking at blebbing brought on by amine- and arginine-reactive compounds from this angle, the residues K71 and R72 are the closest candidates in the primary sequence.

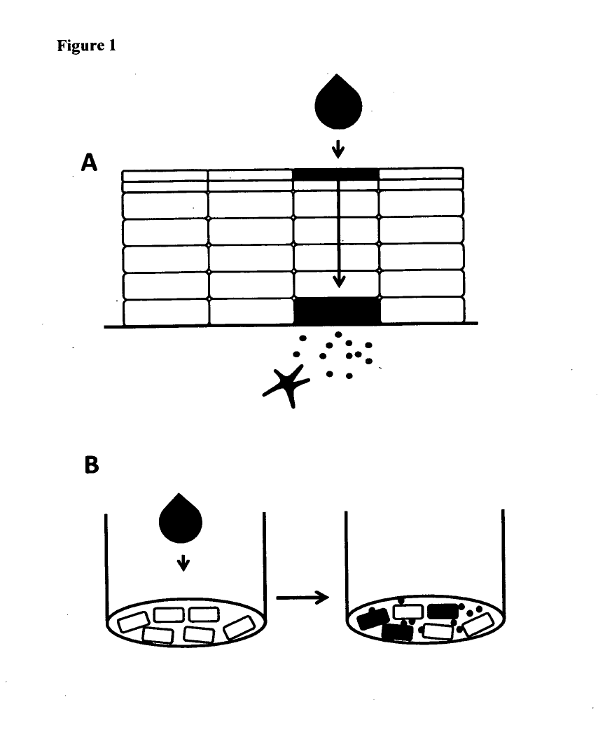

The third step is that this residue (C54) is situated in a stretch of K5 that has been shown through its interactions with desmoplakin, to be vital in maintaining the integrity of the desmosome. It is known that epithelial cells with a compromised cell-cell (and/or cell-matrix) contact undergo apoptosis (“anoikis”). The blebbing that is the result of contact with sensitizing compounds seems to share at least some mechanisms with the naturally occurring apoptosis. Basal keratinocytes consists of up to 35% of insoluble keratin intermediate filaments (KIFs). As basal keratinocytes occasionally undergo apoptosis, a mechanism of waste removal of the debris of insoluble keratins has evolved. In the body, apoptotic basal cells package their remnants in so called “keratin bodies”, that have been shown to consist to a large degree of K5 and K14. The keratin bodies bleb off the cells. Thus, when the keratinocytes are exposed to sensitizing compounds, at least some of the mechanisms behind the naturally occurring apoptosis happen, and KIFs (and other cellular remnants) are packaged in blebs. This step is thus that haptenation provokes the cells to produce blebs (FIG. 1), which has been manifested by the in vitro production of fluorescent, bimane-containing blebs after exposure of keratinocytes to bimanes. This did not occur when the non-reactive, non-allergenic substance synBim was added to the cells. Non-fluorescent, clinically relevant haptens such as oxazolone, formaldehyde, CDNB (1-chloro 2,4 dinitrobenzene), glyoxal, etc., thus provoke a blebbing response. The blebs resulting from addition of oxazolone (predominantly amine-reactive) and glyoxal (predominantly arginine-reactive) also contain keratins.

In SDS-PAGE experiments it was shown that thiol-, amine-, and arginine-reactive sensitizers give rise to blebs that contain the same proteins. In this step, we thus show that by adding haptens to keratinocytes, the haptens induce expulsion of a high amount of cryptic epitopes that are normally concealed from the immune system through their intracellular location and/or neoepitopes consisting of haptenated/differently processed peptides and proteins. It was also shown, using a LTQ-FT-ICR mass spectrometer equipped with a nanospray source, that the blebs contain other proteins besides the mentioned keratins, for example glucose-6-phosphatase isomerase, transitional endoplasmic reticulum ATPase, protein disulfide isomerase, calmodulin, importin 5, keratin 6 (A, B and C), stathmin, transgelin-2, 14-3-3 protein sigma, calreticulin, endoplasmin, heat shock protein 90, actin beta, actin gamma, alpha actinin, cofilin 1, ezrin, fibronectin, myosin 1c, plastin 2, plastin 3, tubulin beta 2C, annexin 2, peroxiredoxin 1, SSA/Ro ribonucleoprotein, alpha enolase, peptidyl-prolyl cis-trans isomerase A. Furthermore, it was found that neoepitopes (hapten-protein conjugates) are expelled (as exemplified by the verification of the C54-hapten conjugate in the blebs). It was also shown that the sensitizing compounds also modify other proteins and that these proteins are released in blebs, as visualized by fluorescent bands on SDS-gels. The neoepitopes concept is further corroborated by the results that bidentate haptens (such as dBBr) cause cross-links between for example keratins.

In addition to the formation of neoepitopes, other problems ensue from cross-linking such as altered protein conformations/processing/clearance. In line with this theory, it can be seen that some sensitizing compounds (including, but not limited to, glyoxal, CDNB (1-chloro-2,4-dinitrobenzene and dBBr (dibromobimane)) result in increased levels of acute-phase proteins such as serum amyloid P component (SAP). Normally, the plasma concentration of SAP increases in response to inflammation. It is thus likely that some sensitizing molecules instigate a perpetual inflammatory response. SAP is highly resistant to proteolysis and its binding stabilizes amyloid fibrils, enhances their formation in vitro, and contributes to their pathogenic deposition and/or persistence in vivo in systemic amyloidosis, autoimmunity and Alzheimer's disease. It is known that chronic inflammation can lead to self-attack (autotoxicity) due to the innate immune defense, and the prominent role of inflammation in Alzheimer's is getting more and more attention. It has also been suggested that SAP may cross the blood-brain barrier, and it is directly neurocytotoxic. SAP has been found to bind to keratin bodies in human skin as well as to isolated KIF aggregates in vitro.

Furthermore, the amount of anti-SAP antibodies in SLE correlates with the disease activity. SAP can also be found in urine. The finding of an increased amount of SAP in the sera of mice exposed to some sensitizers but not others (including, but not limited to, oxazolone) can be utilized as an additional diagnostic in vitro feature.

We also have shown that mice that have been topically exposed to haptens (including mBBr, dBBr, CDNB, oxazolone and glyoxal, not excluding others) have increased levels of anti-nuclear antibodies (ANA) in their sera. Upregulated levels of ANA are commonly used markers for various autoimmune diseases i.e. SLE, RA, SS.

The fourth step is that it has been shown that in the body, keratin bodies drop below the basement membrane. It has been shown that keratin bodies are devoured by antigen-presenting cells. In the dermis, these are chiefly the dermal dendritic cells. Epidermal antigen-presenting cells such as the Langerhans cells are of course also exposed to these cellular remnants, and these cells are in all likelihood also part of the sensitization process. In this step, the neo- and cryptic epitopes are thus devoured by antigen-presenting cells. To demonstrate that keratins are released and function as antigens, ELISA capture of autoantibodies has been performed against keratin in mice that have been exposed, through skin contact, to haptens. The results show that haptens expose keratin to the immune system, and that the protein fragments are antigenic since mice develop autoantibodies against them. This also corroborates that other, less abundant proteins that were found in the blebs (for example stathmin and calmodulin) are released in the body upon contact with sensitizers.

These steps are true also for haptens that preferentially react with other amino acids than cysteine. This was shown by adding a panel of haptens to human keratinocytes, and visualized the cellular response. Haptens, as opposed to the vehicle (DMSO) instigate blebbing. Non-allergenic compounds such as synBim, or nonanoic acid do not induce blebbing. The irritant compound SDS does not cause blebbing, instead the cells seem to “dissolve” when exposed to SDS.

The blebs contain keratins and other proteins. That the blebbing is a general response that also occurs in the body has been demonstrated by capturing autoantibodies against K14 in mice that have been exposed to sensitizing compounds directed at diverse nucleophiles (amino acids) that react through different reaction mechanisms with the same nucleophiles that are monodentate or polydentate. It is envisioned that by including an activation step prior to addition of the compound to the cells (for example activation by phase I detoxication enzymes), prohaptens can be transformed into reactive form(s). As prehaptens are activated outside the body, they function in an analogous fashion to “traditional” haptens.

These steps 1-4 establish sensitization. An in vitro test based on these discoveries is the core of the invention herein.

Through a number of experiments, it was thus found that prominent targets for haptens in human tissue are keratinocytes. It is surprising that, even though the epidermis consists of ˜95% keratinocytes or remnants (corneocytes) thereof, the role of keratinocytes has been overlooked. The stratified nature of the skin allows haptens to reach basal keratinocytes that contain large amounts (up to 35% of the total protein content of the cells) of keratins 5 and 14 (K5 and K14).

The invention is thus based on the discovery that keratinocytes that have been subjected to compounds that are sensitizers for e.g. ACD, produce blebs, and that these blebs contain proteins such as keratins that play an important role in establishing immunity. Other less abundant proteins also found in the blebs (e.g. stathmin and calmodulin) are also released in the body upon contact with sensitizers. The measurement of different aspects of the blebbing such as time to blebbing using comparison to a standard sensitizer, the concentration of the compound at which the keratinocyte cells produced blebs after incubation, the size of blebs at a high concentration of the compound, number of blebs at a high concentration of the compound, the amount of released proteins, such as K5 and K14, in blebs, is thus the basis of the invention. The blebs can be separated from cells through centrifugation, ultracentrifugation and fluorescence-activated cell sorting as is known in the art. The cells used in the invention can e.g. be cultured in multi-well plates, on cell-culture dishes, in test tubes, in flasks, on microscope slides and in bioreactors.

Basal cells in stratified squamous epithelia express K5 and K14 in high abundance, up to 25-35% of the cellular content. The described mechanisms (STEPS 1 through 4, vide supra) are therefore also likely to play a role in ailments whereby sensitization and elicitation are independent of the route of entry of the substance into the body such as through ingestion, eye contact, through inhalation, etc. Thus, if a compound causes blebbing of keratinocytes there is a high risk that the compound in question also can induce other conditions besides ACD. The blebbing response of the invention can therefore also be utilized as a predictive test for other conditions that can be caused by epithelial contact with a compound. These conditions include chemically-induced asthma but do not exclude other ailments.

Furthermore, the finding that the identical or highly homologous epitopes are encountered irrespective of the route of entry of the compound into the body (exemplified by K5/K14 and K8/K18) has other implications. An example of this is DHR where the blebbing response also could be important. Another object of the invention is therefore to use the blebbing response to gauge the risk of compounds (neat, in mixtures, formulations, solutions, etc.) to cause drug-induced hypersensitivity reactions (DHRs). The present invention thus represents a major advancement in predictive screening for adverse drug reactions upon epithelial contact. The test can also be used to screen for inhibitors of the blebbing response in relationship to these effects. First, the assumed inhibitor is added to the cells in the in vitro test in the same way as for sensitizers. No blebs should be formed at the same time-scale or concentrations as for extreme, strong, moderate and weak sensitizers. If the assumed inhibitor passes this first test, i.e. no blebbing response is recorded, it is transferred to the next step. The next step is to incubate the cells in the in vitro test with sensitizers of different strength together with the inhibitor. The blebbing response is evaluated and compared to the blebbing response that resulted from the sensitizers in their selves. The inhibitor is considered to be successful if it decreases or completely prevents the blebbing response.

It is therefore proposed that once blebbing is instigated, and the immunological threshold is passed, self-tolerance is breached. These breaches of tolerance include several targets including K5, K14, and epitopes spreading through homology between released proteins and other self-proteins (for instance between K5 and K8 and K14 and K18). Thus, the invention presented herein can use the blebbing response to measure the ability of compounds, mixtures, solutions and other formulations to cause breach of self-tolerance and thus instigate autoimmune diseases. The test can also be used to screen for inhibitors of the blebbing response in relationship to these effects.

It can thus be concluded that sensitizing compounds, in general, prompt keratinocytes to produce blebs. This corresponds to a key gate in a logistic chain of events that ultimately leads to sensitization (ACD); and/or autoimmune diseases; and/or other allergies; and/or primes the individual for DHRs. The specific outcome in each case is likely to depend on the immune system of the individual and his/hers environmental cues. Several workable parameters have been identified that permitted the development of a graded predictive in vitro test that reflects regulatory issues. Also, the mixture, solution, formulation, etc. can be used after dilution/dissolving to test the combined effects of the compounds in that particular preparation. As the same epitopes are released irrespective of which sensitizing compound is released, there are additive and potentially even synergistic effects, not considered before in the legislation. This has great implications upon how to test compounds and how to estimate safe limits, as the majority of the consumer products contain many sensitizing compounds in the same formulation (for example in perfumes, shampoos, skin lotions, etc.).

One key feature of the murine LLNA, the OECD approved test for ACD that is soon to be obsolete, is that it gives a graded read out where compounds are classified as extremely, strongly, moderately, weakly or non-allergenic. The grading provides an immunotoxicological basis for the legal handling and classification of compounds. This is a feature that is addressed in the cell-based in vitro test of the invention. A mixture of responses to afford a sufficient dynamic range to allow a graded readout is used. Examples of this include (but do not exclude other variables): at what time post-hapten addition does blebbing occur at the different concentrations of hapten? At what concentration does blebbing occur (fixed time-point)? How many blebs are released into the surrounding medium at concentration x and time y? Standardization will be afforded through comparisons with cells that have been subjected to treatment with non-allergens and non-irritants such as for example nonanoic acid or similar compounds. The control samples have not been subjected to any test compounds. The cells could be human primary keratinocytes, cell lines derived from keratinocytes or other epithelial cells that share the necessary blebbing response. The measured parameters will form a matrix where the combined signal will give a graded readout.

Another key feature that is present in the LLNA but that has been overlooked in other cell-based attempts is that of calibration (and repeatability). Typically, HCA (α-hexyl cinnamic aldehyde) is used to calibrate the response and give a check-up of the status of the testing procedure. An HCA calibration experiment is typically included occasionally throughout the year. In the test of the present invention, calibration will be afforded by adding controlled substances that are known to give a specified and reliable response, examples include oxazolone, 1-chloro-2,4-dinitro benzene, mBBr, and HCA, but do not exclude other substances. This calibration can be run every time the test is run. This is in analogy with how a pH electrode is calibrated before each pH measurement. Thus, the test of the invention will actually provide a better calibration than the soon to be obsolete LLNA.

As was previously mentioned, the invention presented herein includes the use of primary human keratinocytes, keratinocytes from other mammals and other relevant mammalian epithelial cells that share the characteristics of the cells (i.e. blebbing when in contact with a hazardous compound. The invention also includes cell lines that share the characteristics of the cells (i.e. blebbing when in contact with a hazardous compound). The invention also includes the use of other cells/cell lines that express keratins and/or mutants thereof. The invention also includes the use of keratinocytes differentiated to a stratified tissue. It is conceivable that the chemical modification of the keratins and the other proteins are such profound events that these proteins or mutants thereof or peptides with sequences based on these proteins could provide a means of gauging the sensitizing capacity of a compound, mixture, solution or formulation. That is, a test could potentially be set up without cells, directly measuring the reactions between the relevant proteins/peptides and the compound(s) in question.

The output measures of the cell-based assay (the blebbing response when a new compound (neat, in mixtures, solutions and other formulations) is added to the cells) can be quantified using various methods, including but not limited to:
i. The amount of released protein (e.g. K5 and K14 or other proteins) through SDS-PAGE and western blots.
ii. The size and amount of released blebs using light microscopy coupled to a digital camera.
iii. The amount of released blebs using membrane probes, for example FM 1-43 (Molecular Probes). This probe is non-fluorescent in aqueous medium, but when inserted in the cell membrane (or bleb-membrane) it turns intensely fluorescent. The amount of fluorescence correlates with the strength of the hapten, i.e. the stronger the hapten, the higher the fluorescence.
iv. The size and amount of released blebs using fluorescence-activated cell sorting. The blebs are sorted according to size and their content can be subsequently analyzed using SDS-PAGE and/or western blots.
v. Flow cytometry systems to count absolute bleb number and to measure the bleb size e.g. Guava Personal Cell Analysis-96 (PCA-96) System (Millipore).
vi. Lab-on-a-chip. “Lab-on-a-chip” or “Microfluidic device” is a device consisting of transparent material suitable for optical microscopy, most usually, but not limited to polydimethylsiloxane (PDMS). The device is only millimeters to a few square centimeters in size. The term also refers to mechanical flow control devices like pumps and valves or sensors like flow meters and viscometers that are connected to the actual device. Within the device are micrometer sized chambers and channels, which have the function of integrating several laboratory steps such as cell culturing, cell sorting and exposing single, or few, cells to different compounds. Number and time scale, as well as size of blebs emerging from a single, or few, cells are evaluated with this method.
vii. High throughput screening. This method is used for rapid in vitro screening of large numbers of compounds using robotic screening assays, combined with, but no limited to imaging devices and protein analysis. In HTS, many compounds are preferably tested on the cells in parallel and screened simultaneously. The image analysis can generate numeric data from the images for many different parameters, such as bleb number, bleb size and fluorescence intensities in labeled cell constituents.
viii. Engineered fluorescent systems that report on the release of blebs. K5-GFP (green fluorescent proteins or similar proteins that are for example fluorescent) transformed cells (e.g. normal human epidermal keratinocytes (HEKn) or cell lines that share necessary HEKn characteristics) are used. When the test compound is added, and if it is a sensitizer, it results in bleb formation, and since K5 is released in the blebs, the blebs will be fluorescent and the fluorescence in the medium can be quantified. Other protein constructs can also be used, for example K14-GFP constructs.
ix. Fluorescent labeling techniques such as e.g. fluorescent K5 and K14 constructs that exhibit fluorescence energy transfer phenomena either with each other or with for example desmoplakin or other proteins that are in close spatial contact may also be used to report on the release of protein in the blebs. If desmoplakin and K5 are separated spatially, then the fluorescence energy transfer (FRET) phenomenon is no longer displayed.
x. Capture systems (for example ELISA) based on antibodies or binders against for example K14 and K5 could also be used to quantify the amount of specific protein released by the cells into the surrounding medium.
xi. Quartz crystal microbalance techniques
xii. Surface plasmon resonance using for example antibodies against the proteins in the blebs as capture reagents.
xiii. Since the cells release blebbing on a timescale that is compatible with that of apoptosis and since the bleb-releasing cells seem to share at least some features with apoptotic cells, labels and kits that are used to measure apoptosis (e.g. annexin V or caspases kits) could also be used to measure the blebbing response with e.g. immunofluorescence and flow cytometry.
xiv. Cells (e.g. HEKn cells or cell lines with HEKn characteristics) can be grown on a semi-permeable support and that blebs and/or bleb content can be harvested by using the “flow-through”. Examples include, but are not limited to using Transwell® Permeable Supports (Corning Life Sciences). The compound (neat, in mixtures, solutions and other formulations) to be tested is added to the surface, the flow-through collected and the amount of released protein (i.e. the blebbing response from the cells) is measured. In an analogous fashion, a support matrix can also be used for growing the cells.
xv. In vitro epithelial tissue that shows the necessary barrier characteristics and penetration characteristics containing cells that show the blebbing response can be used in the analysis. The compound (neat, in mixtures, solutions and other formulations) to be tested is added to the surface of the tissue, the flow-through collected and the amount of released protein (i.e. the blebbing response from the cells in the tissue) is measured.

Further aspects of the invention encompass a kit for the screening of a chemical compound or mixtures, formulations, and solutions thereof, for sensitizing properties, said kit comprising

i) one or more keratinocyte cell cultures
ii) means for growing the one or more keratinocyte cell cultures to confluency,
iii) means diluting the compound to be tested to a selected range of dilutions
iv) instructions to use the means in ii) and iii) according to any of the methods described herein.

Further, said kit may include one or more standard sensitizers as described above.

Further, said kit may include instructional materials disclosing, for example, use of the means for diluting the compound to be tested to a selected range of dilutions and means for growing a keratinocyte culture to confluency, or means of use for a particular reagent. The instructional materials may be written, in an electronic form (e.g., computer diskette or compact disk) or may be visual (e.g., video files). The kits may also include additional components to facilitate the particular application for which the kit is designed. Thus, for example, the kit can include buffers, growth medium, supplements and other reagents routinely used for the practice of a particular disclosed method. Such kits and appropriate contents are well known to those of skill in the art.

The kit may further comprise, in an amount sufficient for at least one assay, the means for assessing the keratinocyte blebbing response described herein to as one or more separately packaged reagents, as well as separate instructions for its use.

Instructions for use of the packaged reagent are also typically included. Such instructions typically include a tangible expression describing reagent concentrations and/or at least one test method parameter such as the relative amounts of reagent and compound to be mixed, maintenance time periods for reagent/sample admixtures, temperature, buffer conditions and the like.

The kit may further include a carrier means, such as a box, a bag, a satchel, plastic carton (such as moulded plastic or other clear packaging), wrapper (such as, a sealed or sealable plastic, paper, or metallic wrapper), or other container.

In some examples, kit components will be enclosed in a single packaging unit, such as a box or other container, which packaging unit may have compartments into which one or more components of the kit can be placed. In other examples, a kit includes one or more containers, for instance vials, tubes, and the like that can retain, for example, one or more biological samples to be tested.

Other kit embodiments include, for instance, syringes, cotton swabs, or latex gloves, which may be useful for handling, collecting and/or processing a biological sample. Kits may also optionally contain implements useful for moving a biological sample from one location to another, including, for example, droppers, syringes, and the like. Still said kit may include disposal means for discarding used or no longer needed items (such as subject samples, etc.). Such disposal means can include, without limitation, containers that are capable of containing leakage from discarded materials, such as plastic, metal or other impermeable bags, boxes or containers.

Kit components may further include packaging necessary for different storage and transport temperatures, such as dry ice for keratinocyte cell cultures or medium supplements or ice packs for components which should be kept cool, e.g. growth medium.

### Example 1

### In Vitro Screening of Chemical Compounds for Risk Assessment in ACD. Analysis Using Light Microscopy

### Cell Culturing

Normal human epidermal keratinocytes (HEKn, Cascade Biologics, Portland, Oreg., USA) were maintained in phenol red-free EpiLife keratinocyte medium (Cascade Biologics) supplemented with 60 μM CaCl₂, the antibiotics gentamicin/amphotericin and 1% (v/v) human keratinocyte growth supplement (HKGS; Cascade Biologics). Alternatively, Medium 154 containing 200 μM CaCl₂ supplemented with HKGS (1% (v/v), without antibiotics is used. Cells were grown at 37° C. in a humidified 5% CO₂ incubator. Final concentrations of the components in the supplemented medium were: bovine pituitary extract, 0.2% v/v; bovine insulin, 5 mg/mL; hydrocortisone, 0.18 mg/mL; bovine transferrin, 5 mg/mL; human epidermal growth factor, 0.2 ng/mL; optional: gentamicin, 10 μg/mL and amphotericin B, 0.25 μg/mL. All reagents were obtained from Cascade Biologics, Portland, Oreg., USA. The keratinocytes were used for experiments in the fifth or sixth passage.

### Screening

HEKn cells in passage 6 were split and resuspended to 18500 cells/ml in medium 154 (Cascade Biologics) supplemented with 1% (v/v) human keratinocyte growth supplement (HKGS; Cascade Biologics, no antibiotics added). Cell suspension (0.5 ml) was added to each well in a 24 well plate (Nunc Multidishes Nunclon, Sigma Aldrich, Stockholm, Sweden) and incubated at 37° C., 5% CO₂. The medium was replaced every second day by fresh 154 medium supplemented with HKGS (no antibiotics added) until cells had grown confluent.

The compounds to be tested (for example: mBBr, SDS, CDNB, nonanoic acid, HCA, oxazolone were purchased from Sigma Aldrich, qBBr, dBBr, glyoxal, and formaldehyde were obtained from Merck-Schuchardt, Darmstadt, Germany) were diluted in DMSO (Sigma Aldrich, Stockholm, Sweden) to final concentrations of 50 mM, 5 mM and 0.5 mM. These stock solutions were prepared fresh each time the experiment was performed. Alternatively, the mixture, solution, formulation, etc. can be used after dilution/dissolving in DMSO (to test the combined effects of the compounds in that particular preparation). As the same epitopes are released, there are additive effects, not considered before in the legislation.

1) Cell preparation just prior to starting the experiment: the medium in the cell culture plate was replaced by 250 μl fresh Medium 154 (Cascade Biologics) supplemented with HKGS (1% (v/v) human keratinocyte growth supplement (HKGS; Cascade Biologics)) and the cells are kept at 37° C., 5% CO₂.

2) Preparation of test compound in medium: 245 μl Medium 154 was added to 0.5 ml microtubes and preheated to 37° C. (waterbath). 5 μl of stock solution of compound was added to the microtubes containing preheated medium. These solutions were then added immediately to the cells in the 24-well plate.

3) The solutions in the microtubes (i.e. 250 μl medium+test compound) were added to each plate. The final concentration of each compound in the test was thus 0.5 mM, 0.05 mM, and 0.005 mM. The plate containing cells and test compounds was incubated for 24 hours at 37° C., 5% CO₂.

4A) Each well was examined with a light microscope to determine the blebbing response (a Nikon TE300 inverted microscope) coupled to a digital camera. The amount of produced blebs at given time points (1.5; 2.5; 24 h) was assessed through visual inspection in blind tests (i.e. the microscopist that counts the blebs had no knowledge of what compound was added to each well). The measured parameters were:
Time to blebbing: Extreme sensitizers (including oxazolone)
   Oxazolone 1.5 h
   Strong sensitizers (including dBBr, mBBr, CDNB)
   mBBr 2.5 h
   CDNB 24 h
Concentration at which the cells produced blebs after 24 hours:
   Extreme sensitizers (including oxazolone)
   0.5 mM, 0.05 mM
   Strong sensitizers (including. dBBr, mBBr, CDNB)
   0.5 mM (dBBr also at 0.05 mM)
   Moderate sensitizer (including glyoxal)
   0.5 mM
   Weak sensitizer (including HCA)
   No blebbing at tested concentrations
Amount of blebs (at time 24 h and concentration 0.5 mM):
   Extreme sensitizers (including oxazolone)
   High amount of blebs (+++)
   Strong sensitizers (including dBBr, mBBr, CDNB)
   Moderate amount of blebs (++)
   Moderate sensitizer (including glyoxal)
   Low amount of blebs (+)
   Weak sensitizer (including HCA)
   No blebs detected

Using these three endpoints a collective analysis of the sensitizing potential of the compounds was made. Oxazolone was found to be an extreme sensitizer, mBBR and CDNB strong sensitizers, glyoxal a moderate sensitizer and HCA did not induce blebbing at the tested concentrations. These results correlated well with LLNA experiments and demonstrated that the method works well for determining the sensitizing properties of compounds.

### Example 2

### In Vitro Screening of Chemical Compounds for Risk Assessment in ACD. Analysis by Fluorescent Dyes

This example is performed as stated in Example 1, except that 24 h after the addition of test compound (exemplified by oxazolone), 3×90 μl is transferred from each well (concentrations 0.5 mM, 0.05 mM, and 0.005 mM) in the 24-well plate to a 96-well plate (Nunc, F96 multisorp) for the measurement of fluorescence to quantify differences in hapten-induced “blebbing” i.e. the strength of the hapten. 10 μl (final concentration 5 μg/ml) of the dye FM 1-43 (T3163, Molecular Probes, Invitrogen AB, Stockholm, Sweden) is added to each well. The plate is incubated in the dark for 2 minutes and the fluorescence is detected in a plate reader (SpectraMax M2, Molecular Devices, Göteborgs Termometerfabrik, Göteborg, Sweden) with excitation at 510 nm and emission at 626 nm.

As the dye FM 1-43 is virtually non-fluorescent in aqueous medium but gets intensely fluorescent when inserted in the cell membrane (or bleb-membrane), it can be used to measure the amount of bleb-membrane in a solution. Thus, the fluorescence correlates with the strength of the hapten, i.e. the stronger the hapten; the more blebs are released into the surrounding media, and the higher the fluorescence in that media.

### Example 3

### In Vitro Screening of Chemical Compounds for Risk Assessment in ACD. Analysis of Released Proteins

The quantification of the blebbing response was also made by observing: the amount of released protein (e.g. K5 and K14) through SDS-PAGE and western blots. Blebs were collected and lysed as described in example 4. SDS-PAGE: The samples were diluted (Blebs: 46 μg, 30 μl for blotting and 29.2 μg, 40 μl for SDS-PAGE. K14 and K5: 0.2 μg, 5 μl) with XT Sample Buffer 4X (BioRad, Hercules, Calif., USA) and milli-Q water. 1 μl DTT (2M) was added to each sample followed by heating at 96° C. for 2 minutes before application on the gel. Electrophoresis program: 200V, 400 mA, 50 W, 50 min. MOPS Running Buffer. The gels were fixed, scanned for fluorescence (excitation 480 nm, emission 530 nm) and coomassie stained (Bio-Safe™ Coomassie, BioRad, Hercules, Calif., USA). The gels are then used for quantitative measurement of proteins. Western Blot: Unfixed gels were blotted onto polyvinyldifluoride membranes (PVDF Ready-gel Blotting Sandwiches, 7×8.5 cm, BioRad, Hercules, Calif., USA). Blotting program: 70 V, 400 mA, 50 W, 30 min. Transfer buffer. Novex XCell II™ Blot Module (E19051, Novex, San Diego, Calif., USA). The blots were incubated in blocking buffer (50 mM Tris-HCl pH 8, 0.2 M NaCl, 3% non fat dry milk), primary Keratin 5 or Keratin 14 Guinea Pig anti-Human polyclonal antibodies (Lifespan Biosciences Inc, Seattle, US) diluted 1:5000 with antibody dilution buffer, and Peroxidase-Rabbit anti-guinea pig IgG (H+ L) secondary antibody (Invitrogen, Carlsbad, Calif., USA) diluted 1:2000 in antibody dilution buffer. The blots were washed 3×10 minutes with wash buffer (50 mM Tris-HCl pH 8, 0.2 M NaCl, 0.05% Tween-20) between each step and developed with DAB and urea peroxide (SIGMAFAST™ 3,3′-Diaminobenzidine tablets, Sigma-Aldrich Chemie Gmbh, Munich, Germany). The peroxidase-DAB reaction was quenched by washing with milli-Q water.

After the development of the blot, the analysis displays whether K5 and/or K14 was present on the SDS-PAGE gel.

### Example 4

### Collection of Blebs

To improve the collection of blebs and avoid contamination by dead, non-adherent cells, this procedure was followed. This procedure results in samples that can be used to measure the blebbing response.

### Cells

Cells (HEKn) were grown in 75 cm² flasks at an initial density of ˜1.8×10⁵ cells/cm² and cultured until the cells reached 70-80% confluency (as stated above).

### Test Compounds

50 mM stock solutions of mBBr, dBBr, qBBr (Sigma Aldrich) and oxazolone (Sigma Aldrich) were prepared in DMSO, glyoxal (Sigma Aldrich) is prepared at 79 mM in Dulbecco's PBS (without Ca, Mg; PAA Laboratories, Linz, Austria). Before incubation, reagents were diluted to their respective concentrations (mBBr, dBBr 0.05 mM; qBBr, glyoxal, oxazolone 0.5 mM) in 5 mL HEPES buffer (140 mM NaCl, 20 mM HEPES, 5 mM KCl, 1 mM MgCl₂, 0.06 mM CaCl₂, 10 mM D-Glucose).

Incubation with Test Compounds

The cell layer was washed with HEPES buffer and incubated with the reagent solutions for 24 hours at 37° C. in a 5% CO₂ humidified incubator (Thermo Forma, Thermo Fisher Scientific, Waltham, Mass., USA).

### Preparation of Blebs

The incubation medium was aspirated from the cell layer and centrifuged at 500×g for 5 min at room temperature to separate blebs from cells that might have lifted off during the incubation period. This step separates dead, non-adherent cells from blebs. The blebs are in the supernatant and the cells are pelleted.

### Further Use of Bleb Solution

The blebs in the supernatant was lysed with 4× freeze-thaw cycles using ethanol, dry ice and a 37° C. water bath. The lysate was concentrated and desalted in 5 kDa MWCO 4 ml Agilent Spin Concentrators for Proteins (Agilent Technologies, Santa Clara, Calif., USA). Total protein concentration was determined with Pierce BCA Protein Assay. The concentrate is stored in −20° C. until analysis of the proteins is performed (according to the method described in Example 3).

### Example 5

### In Vitro Screening of Chemical Compounds for Risk Assessment in ACD. Analysis by Fluorescence-Activated Cell Sorting

In this example, the test procedure is performed as in Example 1 and the collection of blebs is performed as in Example 4. The size and amount of blebs are then analyzed using a flow cytometer with sorting function (FACSAria, BD Biosciences). The blebs are then sorted according to size and each bleb is counted. Beads can be used as standards for size.

### Further Use of Bleb Solution

The collected blebs are lysed and analyzed with respect to protein content as described in example 3.

### Example 6

### In Vitro Screening of Chemical Compounds for Risk Assessment in ACD. Analysis Using Flow Cytometry

This screening is performed as in Example 5. The number of blebs is counted in a flow cytometer (e.g. Guava Personal Cell Analysis-96 (PCA-96) System, Millipore) The size of blebs can be assessed using size standard beads.

### Example 7

### In Vitro Screening of Chemical Compounds for Risk Assessment in ACD Using Lab-on-a-Chip

### Cell Culturing

Cells (HEKn) are grown in 75 cm² flasks at an initial density of ˜4.8×10⁵ cells/cm² and cultured until the cells reached 70-80% confluency (as stated above).

### Lab on a Chip

HEKn cells in passage 6 are split and resuspended to 18500 cells/ml in medium 154 (Cascade Biologics) supplemented with 1% (v/v) human keratinocyte growth supplement (HKGS; Cascade Biologics, no antibiotics added). Cell suspension (0.5 ml) is added to microfluidic device consisting of, but not limited to, e.g., polydimethylsiloxane (PDMS) and incubated at 37° C., 5% CO₂. The medium is replaced by flushing the device every second day by fresh 154 medium supplemented with HKGS (no antibiotics added) until cells have grown confluent.

### Screening

Stock solutions (e.g. 50 mM) of mBBr, dBBr, qBBr (Sigma Aldrich) and oxazolone (Sigma Aldrich) are prepared in DMSO, glyoxal (Sigma Aldrich) is prepared at 79 mM in Dulbecco's PBS (without Ca, Mg; PAA Laboratories, Linz, Austria). Before incubation, reagents are diluted to their respective concentrations (mBBr, dBBr 0.05 mM; qBBr, glyoxal, oxazolone 0.5 mM) in 5 mL HEPES buffer (140 mM NaCl, 20 mM HEPES, 5 mM KCl, 1 mM MgCl₂, 0.06 mM CaCl₂, 10 mM D-Glucose).

Incubation with Test Compounds

The microfluidic device is mounted on optical microscope stage, allowing for transmission light and fluorescence imaging (e.g. Nikon TE300 inverted microscope). The microscope stage is preferably automated which allows for the automatic study of several compounds at the same chip. The device needs to be enclosed in a 37° C., 5% CO₂ humidified incubator. The device containing the cells is flushed with pure HEPES buffer. Thereafter the flow is changed to the test solutions and incubated with the reagent solutions.

### Monitoring Blebs

The cells are continuously monitored (e.g. every 20 minutes) and imaged during the exposure to test solution. The time to onset of blebbing is monitored, as above. But also size and shape of blebs recorded by digital camera connected to the microscope. Simultaneously, the released blebs are collected by the microfluidic dialysis, and collected in microtubes.

The microtubes are centrifuged (as described in example 2) to separate blebs from cells that might have lifted off during the incubation period.

### Further Use of Bleb Solution

The collected blebs are lysed and analyzed with respect to protein content as described in example 3.

### Example 8

### In Vitro Screening of Chemical Compounds for Risk Assessment in ACD Using High Throughput Screening

### Cell Culturing

Cells (HEKn) are grown in 75 cm² flasks at an initial density of ˜1.8×10⁵ cells/cm² and cultured until the cells reached 70-80% confluency (as stated above).

### Screening

HEKn cells in passage 6 are split and resuspended to 18500 cells/ml in medium 154 (Cascade Biologics) supplemented with 1% (v/v) human keratinocyte growth supplement (HKGS; Cascade Biologics, no antibiotics added). Cell suspension (0.5 ml) is added to each well of several 24 well plates (Nunc Multidishes Nunclon, Sigma Aldrich, Stockholm, Sweden) and incubated at 37° C., 5% CO₂. The medium is replaced every second day by fresh 154 medium supplemented with HKGS (no antibiotics added) until cells have grown confluent.

Stock solutions (e.g. 50 mM) of mBBr, dBBr, qBBr (Sigma Aldrich) and oxazolone (Sigma Aldrich) are prepared in DMSO, glyoxal (Sigma Aldrich) is prepared at 79 mM in Dulbecco's PBS (without Ca, Mg; PAA Laboratories, Linz, Austria). Before incubation, reagents are diluted to their respective concentrations (mBBr, dBBr 0.05 mM; qBBr, glyoxal, oxazolone 0.5 mM) in 5 mL HEPES buffer (140 mM NaCl, 20 mM HEPES, 5 mM KCl, 1 mM MgCl₂, 0.06 mM CaCl₂, 10 mM D-Glucose), and added to cells as described in example 1.

The wells are loaded into a HCS device, preferably equipped with a plate loading robot, e.g. Olympus Scan̂R with Hamilton plate loading robot. Both the cell morphology and bleb release will be monitored for a large number of test compounds at different concentrations.

### Further Use of Bleb Solution

The collected blebs are lysed and analyzed with respect to protein content as described in example 3.

### Example 9

### In Vitro Screening of Chemical Compounds for Risk Assessment in ACD Using Engineered Fluorescent Systems

Keratinocytes are transformed to express green fluorescent protein coupled to keratin 5 and keratin 14 according to standard procedures. The test is then performed as in Examples 1 and 4, using these engineered cells instead. The amount of blebs is then analyzed in a plate reader (Spectramax, Molecular Devices Inc., Sunnyvale, Calif., USA), detecting GFP.

### Example 10

### In Vitro Screening of Chemical Compounds for Risk Assessment in ACD. Analysis Using capture systems (ELISA)

The screening is performed as described in example 1. The blebs are separated and lysed according to example 4. The bleb content, e.g. amount of K14, is then analyzed using a sandwich ELISA system:

### Coating

Human anti-K14 capture antibody (Lifespan Biosciences, Seattle, Wash., US) is diluted in PBS (1:500). Fifty (50) μl of the solution is added to the wells in a 96-well plate. The plate is covered with parafilm and incubated in a moist chamber at 4° C. overnight.

### Running

Each well is aspirated and washed with 300 μl wash buffer (TRIS, 0.05% TWEEN) for a total of three washes. After the last wash the remaining wash buffer was removed by invert the plate to clean paper towels. 150 μl blocking solution (0.5% BSA, 0.05M TRIS, pH 7.4) is added to each well and the plate is incubated at room temperature (RT) for 1 hour. The washing steps are performed four times as described earlier.

The bleb content is diluted 1:100, 1:5, 1:5, 1:5 in sample buffer (0.05M TRIS 0.015 M NaCl, pH 7.4) and 50 μl/well are added to the plate in triplicate. Human K14 (Genway Biotech, San Diego, Calif.) is used as a positive control and is prepared in duplicate by serial dilution in sample buffer from 1:400 to 1:6400 in the plate. The plate is incubated at room temperature for 1 hour. The washing steps are performed four times as described above.

Human anti-K14 antibody (Lifespan Biosciences, Seattle, Wash., US) is diluted appropriately (1:3000) and 50 μl is added to each well and the plate is incubated for 30 minutes to an hour at room temperature. The washing steps are performed four times as described above.

The biotinylated detection antibody (Goat polyclonal to mouse IgG, IgM, IgA F(ab)₂ fragment, Abcam, Cambridge, Mass., USA) is diluted 1/1500 in sample buffer and 50 μl is added to each well. The plate is incubated at 37° C. for 2 hours. The washing steps are performed four times as previously described.

Streptavidin-peroxidase is diluted 1:2000 in sample buffer and 50 μl is added to each well and the plate is incubated for 1 hour at RT. The washing steps are performed four times as described above.

A 1 mM ABTS solution is prepared in 70 mM citric buffer (pH 4.2) and protected from light (until use). 10 μl H₂O₂ (30%) is added to 10 ml of ABTS solution. Immediately after the addition of H₂O₂, 100 μl of the solution is added to each well in the plate. The plate is protected from light and incubated for 30 min up to 1 hour. After that, the absorbance is measured at 405 nm in a plate reader (Spectramax, Molecular Devices Inc., Sunnyvale, Calif., USA). The more blebs that are released, the more K14 will be detected by this method.

### Example 11

### In Vitro Screening of Chemical Compounds for Risk Assessment in ACD. Analysis by Measuring Apoptosis

The screening is performed as described in example 1. To detect apoptotic cells ApoDETECT Annexin V-FITC Kit (Invitrogen, Carlsbad, Calif., USA) is used according to the instructions in the manual. The cells are analyzed cells by flow cytometry or fluorescent microscopy. The higher amount of apoptotic cells, the stronger the allergen/sensitizing compound.

### Example 12

### In Vitro Screening of Chemical Compounds for Risk Assessment in ACD Using Cells Grown on Semi-Permeable Support

Keratinocytes are grown as described in example 1, except that the cells are grown on a semi-permeable support instead (Transwell® Permeable Supports with 0.4-8 μm pore size, Corning Life Science). The screening is performed according to example 1. The flow-through is collected with standard pipettes and analyzed with respect to released proteins (e.g. K5 and K14) as described in example 3

### Example 13

### Testing the Capacity of a Compound to Cause DHRs

### Cell Culturing

Normal human epidermal keratinocytes (HEKn, Cascade Biologics, Portland, Oreg., USA) are maintained in phenol red-free EpiLife keratinocyte medium (Cascade Biologics) supplemented with 60 μM CaCl₂, the antibiotics gentamicin/amphotericin and 1% (v/v) human keratinocyte growth supplement (HKGS; Cascade Biologics). Alternatively, Medium 154 containing 200 μM CaCl₂ supplemented with HKGS (1% (v/v), without antibiotics is used. Cells were grown at 37° C. in a humidified 5% CO₂ incubator. Final concentrations of the components in the supplemented medium are: bovine pituitary extract, 0.2% v/v; bovine insulin, 5 mg/mL; hydrocortisone, 0.18 mg/mL; bovine transferrin, 5 mg/mL; human epidermal growth factor, 0.2 ng/mL; optional: gentamicin, 10 μg/mL and amphotericin B, 0.25 μg/mL. All reagents were obtained from Cascade Biologics, Portland, Oreg., USA. The keratinocytes are used for experiments in the sixth passage.

### Screening

HEKn cells in passage 6 are split and resuspended to 18500 cells/ml in medium 154 (Cascade Biologics) supplemented with 1% (v/v) human keratinocyte growth supplement (HKGS; Cascade Biologics, no antibiotics added). Cell suspension (0.5 ml) is added to each well in a 24 well plate (Nunc Multidishes Nunclon) and incubated at 37° C., 5% CO₂. The medium is replaced every second day by fresh 154 medium supplemented with HKGS (no antibiotics added) until cells has grown confluent.

The drug candidates to be tested are diluted in DMSO (Sigma Aldrich) to final concentrations of 50 mM, 5 mM and 0.5 mM. These stock solutions are prepared fresh each time the experiment was performed.

1) Cell preparation just prior to starting the experiment: the medium in the cell culture plate is replaced by 250 μl fresh Medium 154 (Cascade Biologics) supplemented with HKGS (1% (v/v) human keratinocyte growth supplement (HKGS; Cascade Biologics)) and the cells are kept at 37° C., 5% CO₂.

2) Preparation of test compound in medium: 245 μl Medium 154 is added to 0.5 ml microtubes (Eppendorf, Sigma Aldrich, Stockholm, Sweden) and preheated to 37° C. (waterbath). 5 μl of stock solution of drug candidates are added to the microtubes containing preheated medium. These solutions are then immediately added to the cells in the 24-well plate.

3) The solutions in the microtubes (i.e. 250 μl medium+test compound) are added to each plate. The final concentration of each compound in the test is thus 0.5 mM, 0.05 mM, and 0.005 mM. The plate containing cells and test compounds is incubated for 24 hours at 37° C., 5% CO₂.

4A) Each well is examined with a light microscope to determine the blebbing response (a Nikon TE300 inverted microscope). The amount of produced blebs at given time points (1.5; 2.5; 24 h) is assessed through visual inspection in blind tests (i.e. the microscopist that counts the blebs has no knowledge of what compound was added to each well). The measured parameters are:
Time to blebbing: Extreme sensitizers from 1.5 h
   Strong sensitizers 2.5 h-24 h
Concentration at which the cells produced blebs after 24 hours:
   Extreme sensitizers 0.005 mM
   Strong sensitizers 0.05 mM
   Moderate sensitizers 0.5 mM
   Weak sensitizers give no blebbing at tested concentrations
Amount of blebs (at time 24 h and concentration 0.5 mM):
   Extreme sensitizers: High amount of blebs (+++)
   Strong sensitizers: Moderate amount of blebs (++)
   Moderate sensitizers: Low amount of blebs (+)
   Weak sensitizers: No blebs detected

Using these three endpoints a collective analysis of the sensitizing potential (i.e. the capacity to sensitize and/or elicit DHRs) of the compounds is made.

4B) 24 h after the addition of drug candidate, 3×90 μl is transferred from each well (concentrations 0.5 mM, 0.05 mM, and 0.005 mM) in the 24-well plate to a 96-well plate (Nunc, F96 multisorp) for the measurement of fluorescence to quantify differences in drug-induced “blebbing” i.e. the strength of the compound. 10 μl (final concentration 5 μg/ml) of the dye FM 1-43 (T3163, Molecular Probes) is added to each well. The plate is incubated at dark for 2 minutes and the fluorescence is detected in a plate reader (SpectraMax M2, Molecular Devices) with excitation at 510 nm and emission at 626 nm. As the dye FM 1-43 is virtually non-fluorescent in aqueous medium but gets intensely fluorescent when inserted in the cell membrane (or bleb-membrane), it can be used to measure the amount of bleb-membrane in a solution. Thus, the fluorescence correlates with the strength of the compound when, i.e. the stronger the compound; the more blebs are released into the surrounding media (i.e. the more epitopes are released through the actions of the drug candidate), and the higher the fluorescence in that media.

### Example 14

### Testing the Capacity of a Compound to Cause Dhrs Using Other Epithelial Cells

### Cell Culturing

Human Esophageal Epithelial Cells (HEEC, ScienCell Research Laboratories, Carlsbad, Calif., USA), a relevant epithelial cell for DHR, are maintained in Epithelial Cell Medium-2 (EpiCM-2, ScienCell Research Laboratories, Carlsbad, Calif., USA). Cells were grown at 37° C. in a humidified 5% CO₂ incubator. All reagents are obtained from Cascade Biologics, Portland, Oreg., USA.

### Screening

HEEC cells are split and resuspended to 18500 cells/ml in EpiCM-2. Cell suspension (0.5 ml) is added to each well in a 24 well plate (Nunc Multidishes Nunclon) and incubated at 37° C., 5% CO₂. The medium is replaced every second until cells has grown confluent.

The drug candidates to be tested are diluted in DMSO (Sigma Aldrich) to final concentrations of 50 mM, 5 mM and 0.5 mM. These stock solutions are prepared fresh each time the experiment was performed.

1) Cell preparation just prior to starting the experiment: the medium in the cell culture plate is replaced by 250 μl fresh EpiCM-2 and the cells are kept at 37° C., 5% CO₂.

2) Preparation of test compound in medium: 245 μl EpiCM-2 is added to 0.5 ml microtubes (Eppendorf, Sigma Aldrich, Stockholm, Sweden) and preheated to 37° C. (waterbath). 5 μl of stock solution of drug candidates are added to the microtubes containing preheated medium. These solutions are then immediately added to the cells in the 24-well plate.

3) The solutions in the microtubes (i.e. 250 μl medium+test compound) are added to each plate. The final concentration of each compound in the test is thus 0.5 mM, 0.05 mM, and 0.005 mM. The plate containing cells and test compounds is incubated for 24 hours at 37° C., 5% CO₂.

4A) Each well is examined with a light microscope to determine the blebbing response (a Nikon TE300 inverted microscope). The amount of produced blebs at given time points (1.5; 2.5; 24 h) is assessed through visual inspection in blind tests (i.e. the microscopist that counts the blebs has no knowledge of what compound was added to each well). The measured parameters are:
Time to Blebbing
Concentration at which the cells produced blebs after 24 hours
Amount of Blebs

Using these three endpoints a collective analysis of the sensitizing potential (i.e. the capacity to sensitize and/or elicit DHRs) of the compounds is made.

4B) 24 h after the addition of drug candidate, 3×90 μl is transferred from each well (concentrations 0.5 mM, 0.05 mM, and 0.005 mM) in the 24-well plate to a 96-well plate (Nunc, F96 multisorp) for the measurement of fluorescence to quantify differences in drug-induced “blebbing” i.e. the strength of the compound. 10 μl (final concentration 5 μg/ml) of the dye FM 1-43 (T3163, Molecular Probes) is added to each well. The plate is incubated at dark for 2 minutes and the fluorescence is detected in a plate reader (SpectraMax M2, Molecular Devices) with excitation at 510 nm and emission at 626 nm. As the dye FM 1-43 is virtually non-fluorescent in aqueous medium but gets intensely fluorescent when inserted in the cell membrane (or bleb-membrane), it can be used to measure the amount of bleb-membrane in a solution. Thus, the fluorescence correlates with the strength of the compound when, i.e. the stronger the compound; the more blebs are released into the surrounding media (i.e. the more epitopes are released through the actions of the drug candidate), and the higher the fluorescence in that media.

The collective analysis gives information of the sensitizing potential (i.e. the capacity to sensitize and for elicit DHRs) of the compound.

### Example 15

### Testing the Capacity of a Compound to Cause Autoimmune Diseases

### Cell Culturing

Normal human epidermal keratinocytes (HEKn, Cascade Biologics, Portland, Oreg., USA) are maintained in phenol red-free EpiLife keratinocyte medium (Cascade Biologics) supplemented with 60 μM CaCl₂, the antibiotics gentamicin/amphotericin and 1% (v/v) human keratinocyte growth supplement (HKGS; Cascade Biologics). Alternatively, Medium 154 containing 200 μM CaCl₂ supplemented with HKGS (1% (v/v), without antibiotics is used. Cells are grown at 37° C. in a humidified 5% CO₂ incubator. Final concentrations of the components in the supplemented medium are: bovine pituitary extract, 0.2% v/v; bovine insulin, 5 mg/mL; hydrocortisone, 0.18 mg/mL; bovine transferrin, 5 mg/mL; human epidermal growth factor, 0.2 ng/mL; optional: gentamicin, 10 μg/mL and amphotericin B, 0.25 μg/mL. All reagents are obtained from Cascade Biologics, Portland, Oreg., USA. The keratinocytes are used for experiments in the sixth passage.

### Screening

HEKn cells in passage 6 are split and resuspended to 18500 cells/ml in medium 154 (Cascade Biologics) supplemented with 1% (v/v) human keratinocyte growth supplement (HKGS; Cascade Biologics, no antibiotics added). Cell suspension (0.5 ml) is added to each well in a 24 well plate (Nunc Multidishes Nunclon) and incubated at 37° C., 5% CO₂. The medium is replaced every second day by fresh 154 medium supplemented with HKGS (no antibiotics added) until cells has grown confluent.

The compounds to be tested are diluted in DMSO (Sigma Aldrich) to final concentrations of 50 mM, 5 mM and 0.5 mM. These stock solutions are prepared fresh each time the experiment was performed.

1) Cell preparation just prior to starting the experiment: the medium in the cell culture plate is replaced by 250 μl fresh Medium 154 (Cascade Biologics) supplemented with HKGS (1% (v/v) human keratinocyte growth supplement (HKGS; Cascade Biologics)) and the cells are kept at 37° C., 5% CO₂.

2) Preparation of test compound in medium: 245 μl Medium 154 is added to 0.5 ml microtubes (Eppendorf) and preheated to 37° C. (waterbath). 5 μl of stock solutions of test compounds are added to the microtubes containing preheated medium. These solutions are then immediately added to the cells in the 24-well plate.

3) The solutions in the microtubes (i.e. 250 μl medium+test compound) are added to each plate. The final concentration of each compound in the test is thus 0.5 mM, 0.05 mM, and 0.005 mM. The plate containing cells and test compounds is incubated for 24 hours at 37° C., 5% CO₂.

4A) Each well is examined with a light microscope to determine the blebbing response (a Nikon TE300 inverted microscope). The amount of produced blebs at given time points (1.5; 2.5; 24 h) is assessed through visual inspection in blind tests (i.e. the microscopist that counts the blebs has no knowledge of what compound was added to each well). The measured parameters are:

Time to blebbing: Compounds that cause blebbing and thus could potentially cause autoimmune diseases upon epithelial contact: very likely from 1.5 h
Strongly likely: 2.5 h-24 h

Concentration at which the cells produced blebs after 24 hours:
Compounds that are very likely to cause autoimmune diseases upon epithelial contact:
Strongly likely: 0.05 mM
Moderately likely: 0.5 mM
Weakly likely give no blebbing at tested concentrations

Amount of blebs (at time 24 h and concentration 0.5 mM):
Extremely likely: High amount of blebs (+++)
Strongly likely: Moderate amount of blebs (++)
Moderately likely: Low amount of blebs (+)
Weakly likely: No blebs detected

Using these three endpoints a collective analysis of the potency of a compound as regards to causing autoimmune diseases is made.

4B) 24 h after the addition of test compound, 3×90 μl is transferred from each well (concentrations 0.5 mM, 0.05 mM, and 0.005 mM) in the 24-well plate to a 96-well plate (Nunc, F96 multisorp) for the measurement of fluorescence to quantify differences in “blebbing” i.e. the strength of the compound. 10 μl (final concentration 5 μg/ml) of the dye FM 1-43 (T3163, Molecular Probes) is added to each well. The plate is incubated at dark for 2 minutes and the fluorescence is detected in a plate reader (SpectraMax M2, Molecular Devices) with excitation at 510 nm and emission at 626 nm.

As the dye FM 1-43 is virtually non-fluorescent in aqueous medium but gets intensely fluorescent when inserted in the cell membrane (or bleb-membrane), it can be used to measure the amount of bleb-membrane in a solution. Thus, the fluorescence correlates with the strength of the compound when, i.e. the stronger the compound; the more blebs are released into the surrounding media (i.e. the more self-epitopes are released through the actions of the test compound), and the higher the fluorescence in that media.

### Example 16

### Testing the Inhibitory (with Regards to the Blebbing Response) Capacity of a Compound

The compounds to be screened for their inhibitory effects (henceforth called inhibitors) are dissolved in DMSO in a series of dilutions that are appropriate with respect to their final application (i.e. for use in a mixture, solution, formulation together with the compound that normally would cause keratinocytes (or the like) to produce blebs).

### Cell Culturing

Normal human epidermal keratinocytes (HEKn, Cascade Biologics, Portland, Oreg., USA) are maintained in phenol red-free EpiLife keratinocyte medium (Cascade Biologics) supplemented with 60 μM CaCl₂, the antibiotics gentamicin/amphotericin and 1% (v/v) human keratinocyte growth supplement (HKGS; Cascade Biologics). Alternatively, Medium 154 containing 200 μM CaCl₂ supplemented with HKGS (1% (v/v), without antibiotics is used. Cells were grown at 37° C. in a humidified 5% CO₂ incubator. Final concentrations of the components in the supplemented medium are: bovine pituitary extract, 0.2% v/v; bovine insulin, 5 mg/mL; hydrocortisone, 0.18 mg/mL; bovine transferrin, 5 mg/mL; human epidermal growth factor, 0.2 ng/mL; optional: gentamicin, 10 μg/mL and amphotericin B, 0.25 μg/mL. All reagents are obtained from Cascade Biologics, Portland, Oreg., USA. The keratinocytes are used for experiments in the fifth or sixth passage.

### Screening

HEKn cells in passage 6 are split and resuspended to 18500 cells/ml in medium 154 (Cascade Biologics) supplemented with 1% (v/v) human keratinocyte growth supplement (HKGS; Cascade Biologics, no antibiotics added). Cell suspension (0.5 ml) is added to each well in a 24 well plate (Nunc Multidishes Nunclon) and incubated at 37° C., 5% CO₂. The medium is replaced every second day by fresh 154 medium supplemented with HKGS (no antibiotics added) until the cells are confluent.

Oxazolone (i.e. the compound that is causing blebs but which one wishes to include in the drug/shampoo/perfume/dishwasher liquid/etc. (here exemplified by oxazolone, but it could be any compound in any type of formulation or mixture that comes into epithelial contact through use) is diluted in DMSO (DMSO and oxazolone from Sigma Aldrich) to final concentrations of 100 mM, 10 mM and 1 mM. The compounds to be screened for their inhibitory effects (henceforth called inhibitors) are dissolved in the same solution as the hapten in a series of dilutions (0.1 μM to 10 M) that are appropriate with respect to their final application (i.e. for use in a mixture, solution, formulation together with the compound that normally would cause keratinocytes (or the like) to produce blebs). These stock solutions are prepared fresh each time the experiment is performed

1) Cell preparation just prior to starting the experiment: the medium in the cell culture plate is replaced by 250 μl fresh Medium 154 (Cascade Biologics) supplemented with HKGS (1% (v/v) human keratinocyte growth supplement (HKGS; Cascade Biologics) and the cells are kept at 37° C., 5% CO₂.

2) Preparation of test mixture (hapten and inhibitor) in medium: 245 μl Medium 154 is added to 0.5 ml microtubes (Eppendorf) and preheated to 37° C. (water bath). 5 μl of stock solution is added to the microtubes containing preheated medium. These solutions are then added immediately to the cells in the 24-well plate.

3) The solutions in the microtubes (i.e. 250 μl medium+test compound) are added to each plate. The final concentration of each compound in the test is thus 0.5 mM, 0.05 mM, and 0.005 mM. The plate containing cells and test compounds is incubated for 24 hours at 37° C., 5% CO₂.

4A) Each well is examined with a light microscope to determine the blebbing response (a Nikon TE300 inverted microscope) coupled to a digital camera. The amount of produced blebs at given time points (1.5; 2.5; 24 h) are assessed through visual inspection in blind tests (i.e. the microscopist that counts the blebs has no knowledge of what compound was added to each well). The measured parameters are:
Time to blebbing without inhibitor: Oxazolone 1.5 h
An inhibitor should stop keratinocytes from producing blebs when oxazolone (or the new test compound) is added to the cells.
Concentration at which the cells produced blebs after 24 hours:
   Oxazolone 0.5 mM, 0.05 mM
As weak sensitizers and non-sensitizers give no blebbing at (0.5 mM, 0.05 mM, and 0.005 mM; by adding an inhibitor there should likewise be no blebbing.
Amount of blebs (at time 24 h and concentration 0.5 mM):
Extreme sensitizers (like oxazolone) give a high amount of blebs (+++)

As weak sensitizers and non-sensitizers give no blebbing at 0.5 mM, 0.05 mM, and 0.005 mM; an inhibitor should likewise result in no blebbing at time 24 h and concentration 0.5 mM.

Using these three endpoints a collective analysis of the inhibitory potential of the compounds is made. A compound that is efficient at inhibiting the blebbing response when the test compound is added is termed an inhibitor. That inhibitor (or analogs) can then be used together with the test compound in solutions, mixtures, formulations etc., to minimize the risk of causing allergic contact dermatitis/autoimmune diseases/DHRs/chemically-induced asthma, etc.

What is claimed is:

1. A method for screening a chemical compound for sensitizing properties, comprising:
a) diluting the compound to be tested to a selected range of dilutions;
b) adding a selected amount of the dilutions of the compound to a layer of keratinocyte cells grown to confluency;
c) incubating the keratinocyte cells and compound dilutions together;
d) examining the keratinocyte cells to determine keratinocyte blebbing response;
e) quantifying the keratinocyte blebbing response; and
f) using the keratinocyte blebbing response to determine the sensitizing properties of the compound.

2. The method of claim 1, wherein the sensitizing properties of the compound are used to assess risk in allergic contact dermatitis.

3. The method of claim 1, wherein the sensitizing properties of the compound are used to determine the capacity of the compound to cause autoimmune diseases.

4. The method of claim 1, wherein the sensitizing properties of the compound are used to determine the capacity of the compound to inhibit blebbing.

5. The method of claim 1, wherein the sensitizing properties of the compound are used to assess the capacity of the compound to cause drug-induced hypersensitivity.

6. The method of claim 1, wherein the keratinocyte blebbing response is determined using the parameters of: time to blebbing using comparison to a standard sensitizer, the concentration of the compound at which the keratinocyte cells produced blebs after incubation, the size of blebs at a high concentration of the compound, number of blebs at a high concentration of the compound and/or the amount of released proteins such as keratin 5 and keratin 14 in blebs.

7. The method of claim 6, wherein the standard sensitizer is selected from the group consisting of oxazolone, 1-chloro-2,4-dinitro benzene, mBBr, dBBr, glyoxal, formaldehyde and α-hexyl cinnamic aldehyde.

8. The method of claim 1, wherein the blebbing response is quantified by a method selected from the group consisting of: a) determination of the amount and identity of released protein through SDS-PAGE and/or western blots; b) determination of the amount of released blebs using a membrane probe that fluoresces when inserted in a membrane; c) determination of surface plasmon resonance; d) using quartz crystal microbalance techniques; e) using engineered fluorescent systems that report on the release of blebs; f) using fluorescent labeling techniques that exhibit fluorescence energy transfer phenomena; g) using flow cytometry systems to count absolute bleb number; h) using high throughput screening to analyze cell morphology and bleb release; using a microfluidic to analyze the blebbing response; j) using a capture system to quantify the amount of specific proteins; k) using in vitro epithelial tissue that shows the necessary barrier characteristics and penetration characteristics containing cells that show the blebbing response; and l) light microscopy.

9. The method of claim 1, wherein the blebbing response is quantified with a membrane probe, which is non-fluorescent in aqueous medium but becomes intensely fluorescent when inserted in a membrane, by measuring the amount of bleb membrane in a solution using solution fluorescence.

10. The method of claim 9, wherein the membrane probe is FM 1-43.

11. The method of claim 1, wherein the blebbing response is quantified by determining the amount of released keratinocyte protein through SDS-PAGE and/or western blots.

12. The method of claim 11, wherein the released keratinocyte protein is selected from the group consisting of keratin 5, keratin 14, keratin 1, keratin 10, keratin 8, keratin 18, glucose-6-phosphatase isomerase, transitional endoplasmic reticulum ATPase, protein disulfide isomerase, calmodulin, importin 5, keratin 6 (A, B and C), stathmin, transgelin-2, 14-3-3 protein sigma, calreticulin, endoplasmin, heat shock protein 90, actin beta, actin gamma, alpha actinin, cofilin 1, ezrin, fibronectin, myosin 1c, plastin 2, plastin 3, tubulin beta 2C, annexin 2, peroxiredoxin 1, SSA/Ro ribonucleoprotein, alpha enolase, and peptidyl-prolyl cis-trans isomerase A.

13. The method of claim 12, wherein the released keratinocyte protein comprises keratin 5 and keratin 14.

14. The method of claim 1, further comprising after incubation, aspirating liquid from the cell layer and centrifuging so that blebs are in the supernatant and cells that might have lifted off during the incubation period are pelleted; lysing blebs in the supernatant, and quantifying the blebbing response by determining total protein concentration.

15. The method of claim 1, wherein the blebbing response is utilized as a predictive in vitro test for ailments selected from the group consisting of allergic contact dermatitis, drug-induced hypersensitive response, chemically induced asthma, food allergies, and other ailments that can be caused through epithelial contact with a compound.

16. The method of claim 1, wherein the blebbing response is used to gauge the risk of compounds in pure form or in mixtures, formulations, and solutions to cause drug-induced hypersensitivity reactions using relevant human epithelial cells.

17. A product for in vitro screening of compounds, mixtures, formulations, and solutions for sensitizing potential for conditions that can be caused by epithelial contact with a compound, comprising:
a) standardized concentrations of test substances;
b) a keratinocyte cell culture; and
c) at least one standard sensitizer.

18. The product of claim 17, wherein the product screens for a condition selected from the group consisting of risk of allergic contact dermatitis, autoimmune diseases, drug-induced hypersensitivity reactions, food allergies, and autotoxicity.

19. A kit for screening a chemical compound, mixture, formulation or solution for sensitizing properties, comprising:
a) at least one keratinocyte cell culture;
b) means for growing the at least one keratinocyte cell culture to confluent growth;
c) means for diluting the compound, mixture, formulation of solution to a selected range of dilutions; and
d) instructions for using the means for growing the at least one keratinocyte cell culture and the means for diluting.

20. The kit of claim 19, further comprising at least one standard sensitizer.
